(19) United States
(12) Patent Application Publication
(10) Pub. No.: US 2010/0269824 A1
(43) Pub. Date: Oct. 28, 2010

(54) Respirator And/Or Anesthetic Device

(75) Inventors: Harri Friberg, Buchs (CH); Jakob Däscher, Buchs (CH)

Correspondence Address: GIBSON & DERNIER LLP, 900 ROUTE 9 NORTH, SUITE 504, WOODBRIDGE, NJ, 07095, US

(73) Assignee: IMT AG, Buchs (CH)

(21) Appl. No.: 12/740,289

(22) PCT Filed: Nov. 12, 2008
(86) PCT No.: PCT/IB2008/054727

§ 371 Date: May 28, 2010

(30) Foreign Application Priority Data
Nov. 13, 2007 (CH) 01757/07

(51) Int. Cl. A61M 16/01; A61M 16/06; G06F 3/048

(52) U.S. Cl. 128/203.13; 128/205.25; 715/866; 715/707; 715/833

(57) ABSTRACT

A respirator and/or anesthesia device having a display, a computer, at least one memory and a program and at least one input unit, at least two different user profiles being provided in the memory, depending on the type of user and retrievable as needed, having a determining effect on the representation in the display as well as the input options in the input unit.

The invention relates to a respirator and/or anesthesia device, having a display, a computer, at least one memory and a program as well as at least one input unit.

Respirators and/or anesthesia devices are operated by various people when in use. These may include, for example, the physician, an anesthesiologist, the nursing staff and to some extent even the patient himself (in particular in the case of respirators for home use). All these people have different requirements with regard to operation and the information demand associated with the device.

User operation of the respirators and anesthesia devices known so far in the so-called homecare field, in “intermediate care wards” (so-called subacutes), in recovery rooms and in intensive care wards is highly technical and therefore relatively complicated. Through various approaches, operability can be improved somewhat with, for example, larger displays, and different philosophies of operation or as a function of the type of user (physician, caregiver or patient). Unfortunately, operating errors with respirators and anesthesia devices today are still the most common cause of disturbances, injuries and/or in the worst case even fatalities of patients.

The display available for the user interface and the input unit on the devices known so far are in most cases reduced to a screen display, usually consisting of a graphical and or character display as well as input elements such as keys and/or buttons. It has been discovered that the user interface of known devices does not usually take into account the interests of the patient, but instead considers only the interests of physicians and therapists. This means, for example, that patients using a respirator at home without supervision find it very difficult to obtain information about their own recovery status. With the known devices, the patient also does not have the option of adjusting his treatment by making adjustments in the respirator operating settings (i.e., parameters) as needed.

In hospitals today, various types and models of respirators from various manufacturers are generally used. In other words, the user interface on the equipment is fundamentally variable. Physicians and/or therapists must be familiar with many different user interfaces and must adapt to them. This leads to a high training expense in hospitals as well as with the nursing personnel in the homecare field. Furthermore, the risk of operating errors is also increased. Furthermore, only in extremely rare cases are all the functions available on respirators fully utilized because of the complexity of the user interfaces. Therefore, there is also an increased risk of patient treatment errors.

The object of the present invention is to create a respirator and/or anesthesia device which allows individual adjustment of the operability and/or user interface to the respective type of user.

According to the invention, this is achieved by providing at least two different user profiles in the memory, depending on the type of user, so that they can be retrieved and/or made available as needed, these user profiles being critical in determining the representation in the display and also the input possibilities in the input unit. By retrieving the corresponding user profile, the device is adapted individually and optimally to the particular type of user. Due to this high flexibility of the device, it is possible to cover a very large range of users with a single type of device under some circumstances. the number of items can be increased and thus cost savings can be achieved by reducing the range of models and limiting it to a small number of models.

The user profiles may yield a specific basic setting for the particular type of user and treatment case. However, there are considerable differences in requirements from one case to the next when there are a variety of patients to be treated. It is therefore expedient for the respective user profiles to be individually adaptable by the patient within predetermined (allowed) limits. The patient's needs may also change in the course of treatment, so that respiration must be stronger or weaker, for example. It is likewise conceivable that an anesthesia device should be switched from the anesthesia mode to the respiration mode, which may then later also be monitored by the patient himself under some circumstances, after which an anesthesia mode to be monitored by a physician should again be possible under some circumstances. The latter should naturally not be accessible to a patient in order to avoid inadvertent anesthesia.

With the devices known so far, the operating instructions supplied are usually quite extensive. In most cases however, they are difficult to understand and using them is very time consuming. It is therefore advantageous that, with at least one user profile, visual user interface instructions (via display) and/or acoustic instructions (via loudspeaker) are implemented. In case of need as well as for training purposes, the user may thus be guided to the desired information in a very efficient and time-saving manner.

Respirators are mostly used in long-term treatment of patients. A patient's activity will usually vary during the day and at night, so his respiration needs are also variable. It is therefore expedient to provide different respiration profiles and/or user profiles for daytime operation and nighttime operation. Due to the different profiles, optimum adaptation to the patient's needs is possible.

In most cases, the day-and-night rhythm is constant. A timer is therefore advantageously provided for automatic switching from daytime mode to nighttime mode (and vice versa). With the help of a timer that can be programmed for all seven days of the week, it is also possible to take into account irregularities in the course of a week, such as a doctor's visit or the like.

Some patients have an irregular sleep rhythm. In these cases, it is therefore advantageous to provide a switching device operable individually by the patient himself for manual switching from daytime mode to nighttime mode (and vice versa). These patients are thus able to decide on their own when they want to switch the device from daytime mode to nighttime mode (and vice versa).

There are some substantial differences within the different user types. It is therefore advantageous if different user profiles for specific application cases are retrievable for at least one type of user. These user profiles may be partially adapted and stored by an authorized user himself. It is therefore also expedient to provide a user authorization routine with the program, which allows a supervisor to grant certain mode switching rights to certain users.

In some cases, devices from different manufacturers and different models are used in treatment of patients. The diversity in these devices makes very high demands of the operating personnel. This is the case in particular because the user interface philosophy of the individual device manufacturers and/or their choice of symbols is/are often different. Furthermore, there is the risk of operating errors. To prevent this and to facilitate a switch from one device to another by the operating person, the display and/or the input unit is/are advantageously configurable in their function and in their appearance through the software, so they are fully compatible with different peripheral devices (chameleon feature). For certain applications in a certain device environment, desired display representations may be selected with input support by the software.

In many cases, the respirators today do not require direct care by a physician. Many patients therefore miss the confidence-inspiring presence of a physician. Patients feel they are left alone with the device. Therefore, according to a refinement of the invention, it is expedient for the program to include a program part which represents a virtual physician in combination with a specific user profile on the display, guiding the respective user acoustically and visually (multimedially) through the use and/or through the treatment in an interactive procedure or being available to give advice if patients have questions.

In unusual situations and/or emergencies, there is a risk of the patient becoming agitated and anxious. The program therefore advantageously includes another program part, which displays a “smart emergency function” on the screen in conjunction with a specific user profile, guiding the user multimedially in the event of an emergency or as needed through the corresponding user interface on an abbreviated pathway and informing the user in a calming manner. This smart emergency function makes it possible to reassure and calm the patient in a short period of time.

Many patients quickly panic in unusual situations such as dyspnea and then they no longer know what to do to help themselves. For these cases, it is expedient for the input unit to have a panic element which triggers a special user profile in a software-supported operation and/or for a program routine to be provided which establishes an external connection and/or a connection to a central office when the panic element is operated. In case of need, suitable assistance from the central office may thus be summoned immediately or sent to the device interactively. The programming may also be such that, in the event of a panic, the program part with the virtual physician can be activated automatically in addition to the smart emergency function because the simplified user interface, supported by calming and reassuring instructions by the virtual physician, can provide the most efficient help to prevent errors in response on the part of the patient. For example, the panic element may be designed as a mechanically operable switch (e.g., as a pushbutton), as a proximity switch that is operable without contact or as part of a display screen.

For training and further education as well as leisure use, multimedia exercise programs, entertainment contributions or the like may be input and/or played back advantageously via the internal memory or an additional external memory or via a hard drive. These programs are preferably also exchangeable and adaptable to the particular person.

In operation of the device, problems may occur over a period of time. To obtain rapid and clear assistance, it is therefore expedient for a program-supported assistance program to be retrievable directly by the user and any time with each element of the user interface (i.e., display) so that the user is informed and supported immediately and multimedially.

Due to the complexity of the device, numerous connections (sensors, lines, etc.) are provided on the device. These may lead to confusion. To prevent this, information and explanations about all connections on the device can be retrieved at any time by the user in a program-supported operation by operating an information button. The memory preferably also contains a photograph of the various connections, which can be displayed on the screen in the info mode, and the program is equipped so that in the info mode, the respective connections are shown with markings in the photograph with detailed explanations about the connections.

For a clear and compact design and good operability of the device, it is expedient for the measured values and/or setting values of the user profiles and corresponding adjusting elements for these values to be displayable on the screen and/or displayable virtually and for the display screen to be embodied as a touchscreen.

On the one hand, the adjusting elements are advantageously designed as sliders and/or in the form of plus and minus keys, and on the other hand, the display scales are electronically connected to the adjusting elements, so that a user can perform the desired settings by means of the adjusting elements as well as by means of corresponding touch of the display scales.

For simple operation of the device, visualization software is advantageously provided; by means of this software, the size and/or length of the adjusting elements change(s) proportionally on operation of the adjusting elements. The user can thus recognize immediately in which direction the values to be set are being changed.

The respective measured values and/or setting values are expediently displayed in the form of an analog scale. The values displayed may also be represented in the form of bar graphs or histograms.

The scale of the measured values and/or setting values and the adjusting elements advantageously overlap at least partially at the sides. Due to this overlap, there is a clear-cut and simple correlation of the adjusting elements with the corresponding measured values and/or setting values.

The length of the scale imaged for the measured values and/or setting values preferably corresponds approximately to the sum of the lengths of the adjusting elements. When the adjusting elements are arranged next to the scales for the measured values and setting values, the position of the adjusting elements thus corresponds to the respective measured value or setting value.

At least one user profile is advantageously provided, allowing an individual adjustment of the respiration parameters to respiration characteristics of the patient using the respirator or vice versa in individual process steps taking place in succession in multimedia communication with the patient. Special properties may be taken into account in the respiration of the patient (mask fit).

In the user profile described above, at least one or more of the following method steps are preferably provided:
instructing the patient to breathe consciously and deeply without placing the mask on his face;
instructing the patient to place the mask on his face and to breathe through the mask without connecting it to the device;
instructing the patient to connect the mask to the device and to breathe through the mask without starting artificial respiration by the device, in order to detect the respiration parameters by means of sensors inside the device;
automatically adjusting the basic settings of the device to the patient's current needs using the respirator.
instructing the patient to start operation of the respiration and/or anesthesia process.

Due to this “learning program” the respiration parameters of the respirator or anesthesia device are optimally adapted automatically to the needs of the patient. Through the individual steps of the program, uncertainties and feelings of anxiety on the part of the patient can also be largely avoided.

With prolonged respiration of a patient, the patient's needs may change over a period of time in accordance with his prevailing situation. It is therefore advantageous that, if necessary, manual overwriting corrections can be made in the basic settings in accordance with the desires and needs of the patient.

The invention will be explained in greater detail below on the basis of the drawings, which show, for example:

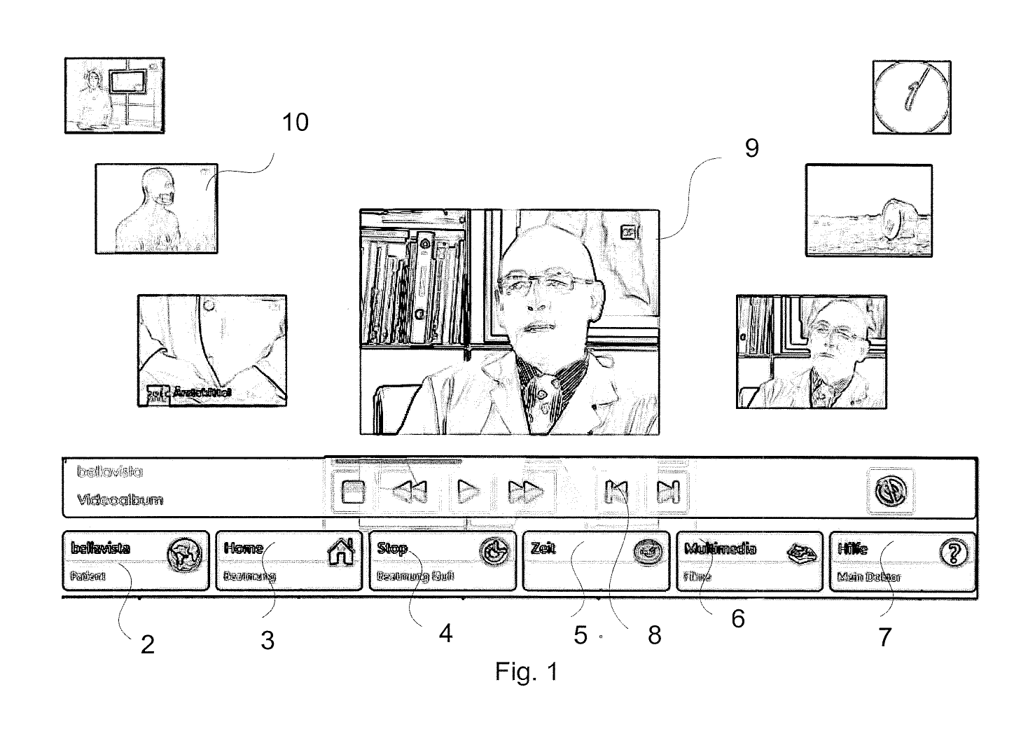

FIG. 1 shows a display of an inventive respirator set for the patient mode;

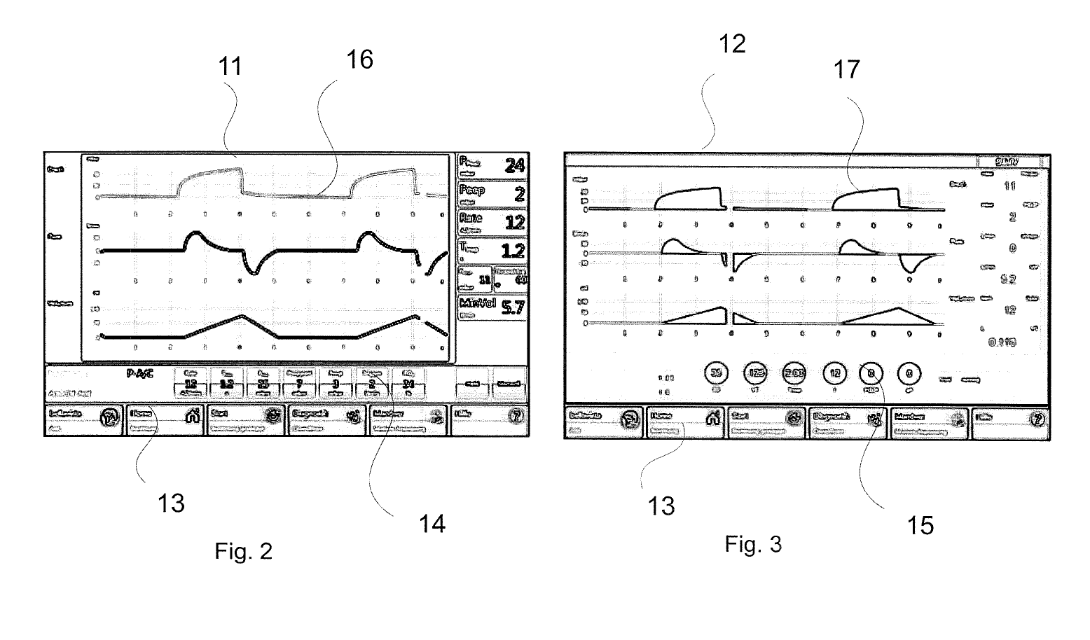

FIG. 2 shows a standard display set at the factory in the setting mode or operator mode; and

FIG. 3 shows a display adapted by the user to the design of existing devices, shown in the setting mode or operator mode.

The list of reference numerals as well as the patent claims are part of the disclosure.

The display, which is labeled as 1 on the whole and can be seen in FIG. 1, is set for “patient mode”. The display 1 is embodied as a so-called touchscreen and is used for the display and for operation of the device. The bottom toolbar of the operating elements contains a user profile selection key 2, a mode selector key 3, a start/stop key 4, an operating and treatment time input key 5, a multimedia selector key 6 and a help and panic key 7. The middle toolbar is embodied as an operating set 8 for the user menu in multimedia mode. A main multimedia image 9 has been inserted in the upper area of the display 1. In the state illustrated here, this main multimedia image 9 shows a virtual physician who provides information for the patient. In the state illustrated here, several secondary multimedia images 10 are also shown on the display 1 in addition to the main multimedia image 9, with the help of which the virtual physician shown in the main multimedia image, for example, gives his explanations. With the help of the operating set, stored films may also be retrieved and played back. The inventive use of audio technology need not be explained in greater detail here because this information is readily available to those skilled in the art with a knowledge of the above inventive idea.

FIG. 2 shows the display in the setting mode and/or operator mode with the standard display of settings. The operating keys 13 in the bottom toolbar are identical to those of the patient mode shown in FIG. 1. The middle toolbar now contains the standard parameter display 14. The parameter curves 16 of the set functions are displayed graphically in the upper field.

FIG. 3 shows the display in the setting mode and/or operator mode with a modified setting display. The operator keys 13 in the bottom toolbar are also identical to those of the patient mode shown in FIG. 1. The middle toolbar now contains an adapted parameter display 15.

The parameter curves 17 in the upper field as well as the parameter display 15 in the middle toolbar are adapted to the display of other devices in use. For the user, this facilitates operation and helps to prevent operating errors. This variable adaptation is also referred to as the so-called chameleon feature.

### LIST OF REFERENCE NUMERALS

1 display (in patient mode)

2 user profile selector key

3 mode selector key

4 start/stop key

5 operating time and treatment time input

6 multimedia selector key

7 help and panic key

8 operating set for multimedia

9 main multimedia image

10 secondary multimedia images

11 standard setting display

12 adapted setting display

13 operating keys

14 standard parameter display

15 adapted parameter display

16 parameter curves

17 parameter curves

1. A respirator and/or anesthesia device, comprising a display, a computer, at least one memory and a program and at least one input unit, wherein at least two different user profiles are provided in the memory, and wherein the profiles are dependent on the type of user, are retrievable on demand and are operable to determine the representation in the display and input options in the input unit.

2. The device according to claim 1, wherein the respective user profiles are operable to be adapted individually by a patient within predefined allowed limits.

3. The device according to claim 1, wherein in the application case with at least one user profile, the device is operable to guide a user visually via display and/or acoustically via loudspeaker.

4. The device according claim 1, comprising different respiration profiles and/or user profiles for daytime mode and nighttime mode.

5. The device according to claim 4, comprising a timer for switching automatically from daytime mode to nighttime mode and vice versa.

6. The device according to claim 4, comprising one or more switching elements operable individually by the patient for manual switching from daytime mode to nighttime mode and vice versa.

7. The device according to claim 1, wherein different user profiles for specific application cases are retrievable for at least one user type.

8. The device according to claim 1, wherein the software is operable to configure the display and/or the input unit in their function and in their appearance, so that they are fully compatible with different peripheral devices.

9. The device according to claim 3, wherein the program contains a program part, which in combination with a specific user profile, shows a virtual physician on the display, guiding the respective user interactively through the application and/or through the treatment acoustically and visually.

10. The device according to claim 1, wherein the program contains a program part which displays on the display screen a smart emergency function in combination with a specific user profile to guide and inform the user multimedially through the corresponding user interface on a shortened pathway in the event of an emergency and/or in case of need.

11. The device according to claim 1, wherein the input unit has a panic element which is operable to trigger a special user profile in a software-supported procedure and/or a program routine is provided which establishes a link to the outside and/or to a central office when the panic element is operated.

12. The device according to claim 1, wherein multimedia exercise programs and entertainment contributions can be input and played back via the memory or an extra external memory or hard drives.

13. The device according to claim 1, wherein a help program is provided which immediately informs and supports the user multimedially, which program can be retrieved directly by the user at any time and at any element on the user display.

14. The device according to claim 1, wherein information and explanations about all the connections on the device can be retrieved by the user at any time by operating an information key.

15. The device according to claim 1, wherein measured values and/or setting values of the user profiles and corresponding adjusting elements for these values may be shown in the display and/or represented virtually and the display is a touchscreen.

16. The device according to claim 15, wherein the adjusting elements are sliders and/or in the form of plus and minus keys, and the display scales are electronically connected to the adjusting elements, so that a user can perform settings by means of adjusting elements and also by touching the display scales accordingly.

17. The device according to claim 16, comprising visualization software, by means of which size and/or length of the adjusting elements is/are varied proportionally when the adjusting elements are operated.

18. The device according to claim 15, operable to display the respective measured values and/or setting values in the form of an analog scale.

19. The device according to claim 18, wherein the scale of the measured values and/or setting values and the adjusting elements overlap at least partially at the sides.

20. The device according to claim 15, wherein the length of the scale imaged for the measured values and/or setting values corresponds approximately to the sum of the lengths of the adjusting elements.

21. The device according to claim 1, comprising at least one user profile, the device operable to allow an individual adaptation of the respiration parameters to the respiration characteristics of the person using the respirator or vice versa in individual process steps taking place in succession and communicated to the patient multimedially.

22. A method for performing respiration and/or anesthesia using a device according to claim 21, wherein one or more of the following method steps are provided in the user profile:
instructing the patient to breathe consciously and deeply without placing the mask on his face;
instructing the patient to place the mask on his face and to breathe through the mask without connecting it to the device;
instructing the patient to connect the mask to the device and to breathe through the mask without starting artificial respiration by the device in order to detect the respiration parameters by means of sensors inside the device;
automatically adjusting the basic settings of the device to the patient's current needs using the respirator; and
instructing the patient to start operation of the respiration and/or anesthesia process.

23. The method according to claim 22, wherein manual overwriting corrections in the basic settings are made as needed in accordance with the wishes and needs of the patient.
